(19) United States
(12) Patent Application Publication
(10) Pub. No.: US 2010/0310660 A1
(43) Pub. Date: Dec. 9, 2010

(54) DRY POWDER MICROPARTICLES FOR PULMONARY DELIVERY

(75) Inventors: Tsuimin Tsai, Taipei (TW); Chin-Tin Chen, Taipei (TW); Jen-Chang Yang, Taipei (TW); Yu-Tsai Yang, Taipei (TW)

Correspondence Address: WPAT, PC;INTELLECTUAL PROPERTY ATTORNEYS, 2030 MAIN STREET, SUITE 1300, IRVINE, CA, 92614, US

(73) Assignee: TAIPEI MEDICAL UNIVERSITY, Taipei (TW)

(21) Appl. No.: 12/480,469

(22) Filed: Jun. 8, 2009

(51) Int. Cl. A61K 9/14; A61K 38/16; A61K 38/02; A61K 31/407; A61P 11/00

(52) U.S. Cl. 424/489; 514/410; 977/773; 977/906; 514/1.1; 514/1.5; 514/185

(57) ABSTRACT

The invention provides a dry powder microparticle for pulmonary delivery, which comprises at least one nanoparticle in the form of liposome or micelle wherein the nanopaparticle encapsulates one or more therapeutic agent therein, and a diluent layer surrounding the nanaparticles.

### FIELD OF THE INVENTION

The invention provides a new platform of dry powder microparticles containing nanoparticle entrapped therapeutic agent therein. Particularly, the nanoparticle contained therein is in the form of micelle.

### BACKGROUND OF THE INVENTION

The route of administration of a drug substance can be critical to its pharmacological effectiveness. Pulmonary drug delivery relies on inhalation of an aerosol through the mouth and throat. Drugs intended for systemic activity can be absorbed into the bloodstream through epithelium cells. Alternatively, if the drug is intended to act topically, it is delivered directly to the site of activity. It has recently been demonstrated that the lung may be an ideal site for non-invasive delivery of drug substances or therapeutic molecules to the systemic circulation. Local delivery of medication to the lung is also highly desirable, especially in patients with specific pulmonary diseases like cystic fibrosis, asthma, chronic pulmonary infections or lung cancer. The lung is an attractive route for drug delivery owing to its enormous surface area for absorption, highly permeable epithelium compared with the gastrointestinal tract, and favorable environment for drugs compared to the low pH and high protease levels associated with oral delivery. In addition, pulmonary drug delivery avoids first pass hepatic metabolism and is generally more acceptable to patients than an injection. To prepare inhalable powders, spray-drying is a common practiced method. Spray-drying has been applied to a variety of substances such as peptides, antibodies, vaccines and carrier particles. U.S. Pat. Nos. 6,610,653, 5,658,878, 5,747,445 and 6,165,976 discloses a therapeutic powder preparation for inhalation comprising insulin and a substance (such as lactose) which enhances the absorption of insulin in the lower respiratory tract. U.S. Pat. No. 6,630,121 provides a method of making fine dry particles of substances by forming a composition comprising a substance of interest and a supercritical or near critical fluid; rapidly reducing the pressure on said composition, whereby droplets are formed; and passing said droplets through a flow of heated gas. U.S. Pat. No. 6,846,801 discloses a method of treating a patient in need of insulin treatment, including the steps of introducing into the lower respiratory tract of the patient a therapeutic preparation in the form of a dry powder containing insulin and an enhancer compound.

Although promising, delivery of therapeutics to the lungs faces several anatomical and physiological challenges. To deposit in the lungs, drugs must traverse a complex lung structure that is heterogeneous in geometry and environment from patient to patient. Once deposited, natural clearance methods, including the “mucociliary escalator”, work to expel particles from the upper airways, while alveolar macrophages rapidly (often within minutes) engulf particles between 1 and 5 Mm that reach the deep lungs. In the area of the tracheo-bronchial region, the epithelium is protected by a mucus layer. Any particle of drug is transported away from the lung by mucociliary clearance. Consequently, larger molecules will not be able to reach their site of drug action. Studies using inhaled nanoparticles dispersed in aqueous droplets suggest that the mucus clearance can be overcome by nanoparticles, possible due to rapid displacement of particles to the airway epithelium via surface energetics. Therefore, nanoparticles may be possible vehicles of transporting drugs efficiently to the epithelium, while avoiding unwanted mucociliary clearance. U.S. Pat. No. 6,811,767 is directed to aerosol formulations of nanoparticulate drug compositions, and methods of making and using such aerosol formulations, in which essentially every inhaled particle contains at least one nanoparticulate drug particle comprising highly water-insoluble drug.

However, there are some problems that using nano-sized delivery systems to overcome for pulmonary delivery is due to their mass medium aerodynamic diameter (MMAD) is not suitable for inhalation delivery. Nano-size carrier was generally too small can easy be exhaled from the respiratory tract. In addition, since such nanoparticles are formed through hydrophobic interaction, the size of these particles exhibits high variation and they might aggregate together in aqueous environment of the lung epithelium so that the solubility of drug decreases. Jeffrey O.-H. et al. investigated the feasibility of developing a platform for aerosol delivery of nanoparticles and showed that nanoparticles were potent drug carriers (International Journal of Pharmaceuticals 269 (2004) 457-467). Shirzad Azarmi et al. provided doxorubicin (DOX)-loaded nanoparticles which were incorporated as colloidal drug delivery system into inhalable carrier particles using a spray-freeze-drying technique (International Journal of Pharmaceutics 319 (2006) 155-161). In the above prior art references, the nanoparticles were prepared with gelatin method using gelatin as carrier (Jeffrey O.-H. et al.) or emulsion polymerization method using n-butylcyanoacrylate as carrier (Shirzad Azarmi et al.). However, the drugs in these nanoparticles may aggregate together and not distribute evenly, so the drugs cannot be completely absorbed by lung, thus reducing their pharmacological activity.

However, there is a need in the art for improved spray-dried powders containing nanoparticles suitable for pulmonary delivery.

### SUMMARY OF THE INVENTION

The invention provides a dry powder microparticle for pulmonary delivery, which comprises at least one nanoparticle in the form of micelle wherein the nanopaparticle entraps one or more therapeutic agent therein, and a water-soluble diluent layer surrounding the nanaparticles.

### BRIEF DESCRIPTION OF THE DRAWING

FIG. 1 shows the photomicrographs at 1000× magnification; (a) optical microscope and (b) fluorescence microscope.

FIG. 2 shows the spray-dried powder morphology visualized by scanning electron microscopy. The spray-dried powders were prepared with L122 micelle Hp/lactose (1:20, wt/wt, lactose: 2%).

FIG. 3 shows the profiles of the absorption spectra of L122 micelle Hp and lactose-L122 micelle Hp after they were dissolved in water.

FIG. 4 shows the oxidation of RNO by singlet oxygen produced by illuminating free Hp (Hp), micelle Hp loaded with L122 micelle (L122 micelle Hp) in PBS and the spray dried Lactose-L122 micelle Hp that was re-dissolved in PBS in the presence of histidine in PBS, measured by loss of absorbance at 440 nm.

FIG. 5 shows the influence of the drug concentration on cellular uptake of free Hp, Hp loaded L122 micelle (L122 micelle Hp) and L122 micelle Hp loaded lactose microparticle (Lactose-L122 micelle Hp). The A549 mammary tumor cells were incubated at different equivalent drug concentrations in DMEM medium for 3 hr. (Mean±SD, n=6).

FIG. 6 shows the comparison of cytotoxicity of A549 cell line after treatment with free Hp (Hp), Hp loaded L122 micelle (L122 micelle Hp) and L122 micelle Hp loaded lactose microparticle (Lactose-L122 micelle Hp) after incubation of 3 hr and followed by illumination for 4, 6, 8 J/cm². (Hp: 0.5 μg/ml).

### DETAILED DESCRIPTION OF THE INVENTION

The invention develops a new platform for dry powder microparticles containing nanoparticle entrapped therapeutic agent therein. The dry powder microparticles can readily dissolve after they reach a trachea and the nanoparticles released therefrom can overcome mucociliary clearance and successfully deliver therapeutic agent to lung (even deep lung) to achieve local or systematic administration. In addition, the therapeutic agent entrapped in the micelle-form nanoparticles will not aggregate and will exist in a monomer form because the molecule of the therapeutic agent individually binds to the polar head or hydrophobic tail of the micelle depending on the hydrophilic or hydrophobic property of the agent.

### A. Dry Powder Microparticle for Pulmonary Delivery of the Invention

The invention provides a dry powder microparticle for pulmonary delivery, which comprises at least one nanoparticle in the form of micelle wherein the nanopaparticle entraps one or more therapeutic agents therein, and a water-soluble diluent layer surrounding the nanaparticles.

As used herein, “dry powder microparticle” refers to a powdered particle that is a finely dispersed solid and is capable of being (i) readily dispersed in an inhalation device and (ii) inhaled by a subject so that a portion of the particles reaches the lungs to permit penetration into the alveoli. Such a powder is considered to be “respirable” or suitable for pulmonary delivery. According to one embodiment of the invention, the dry powder microparticle is of a size ranging from 1 to 10 μm, preferably 1 to 5 μm, 5-8 μm or 8-10 μm W.H. Finlay and M.G. Gehmlich. Inertial sizing of aerosol inhaled from two dry powder inhalers with realistic breath patterns versus constant flow rates. Int J. Pharm. 210:83-95 (2000).

As used herein, “nanoparticle” refers to a particle having a size of less than about 1,000 nanometers; preferably, 3 to 1000 nm (N. K. Jain. Pharmaceutical technology, Pharmaceutical nanotechnology. 17Sep. 2007; P. Couvreur, G. Couarraze, J. P. Devissaguet and F. Puisieux, Nanoparticles: Preparation and Characterization. Microencapsulation. 73:183-211 (1996). According to one embodiment of the invention, the size of the nanoparticle ranges from 3 to 700 nm, 3 to 500 nm, 3-300 nm, 3-150 nm, 3-110 nm, 3-100 nm, 3-50 nm, 3-30 nm, 50-100 nm, 50-200 nm, 50-300 nm, 50-500 nm or 50-700 nm, preferably 3 to 150 nm, more preferably 3 to 110 nm, even more preferably 3 to 50 nm or 3 to 30 nm. According to the invention, the nanoparticle is in the form of liposome of micelle. The shape of the micelle or liposome can vary and can be, for example, prolate, oblate or spherical; spherical micelles or liposomes are most typical.

As used herein, “micelle” shall include “normal micelle” and “reverse micelle”. A normal micelle is a micelle in which the micelle has a hydrophilic outer shell and a hydrophobic inner core, while a reverse micelle is the opposite, i.e., a hydrophobic outer shell and a hydrophilic inner core. Micelle formation occurs as a result of two forces. One is an attractive force that leads to the association of molecules, while the other is a repulsive force that prevents unlimited growth of the micelles to a distinct macroscopic phase. As contemplated herein in one embodiment of the present invention, the micelle has an outer hydrophilic shell and an inner hydrophobic core. Under these circumstances, the linkage between the support surface and the micelle is preferably a covalent bond between the hydrophilic shell and the support surface. Polymeric micelles seem to be one of the most advantageous carriers for the delivery of water-insoluble drugs. Polymeric micelles have many advantages on administration and delivery of drugs such as their small particle size (<200 nm), targeting ability, long circulation time and easy production (J Control Release. 73:137-172m 2001). Polymeric micelles are characterized by a core-shell structure. Pharmaceutical research on polymeric micelles has been mainly focused on copolymers having an X-Y diblock structure with X, the hydrophilic shell moieties and Y the hydrophobic core polymers. Multiblock copolymers such as poly(ethylene oxide)-poly(propylene oxide)-poly(ethylene oxide) (PEO-PPO-PEO) (X-Y-X) can also self-organize into micelles, and have been described as potential drug carriers (FEBS Lett. 258 (1989) 343-345). The hydrophobic core which generally consists of a biodegradable polymer such as a poly(beta-benzyl-L-aspartate) (PBLA), poly (DL-lactic acid) (PDLLA) or poly (epsilon-caprolactone) (PCL) serves as a reservoir for an insoluble drug, protecting it from contact with the aqueous environment. The core may also consist of a water-soluble polymer, such as poly(aspartic acid) (P(Asp)), which is rendered hydrophobic by the chemical conjugation of a hydrophobic drug, or is formed through the association of two oppositely charged polyions (polyion complex micelles). The hydrophobic inner core can also consist of a highly hydrophobic small chain such as an alkyl chain or a diacyllipid such as distearoyl phosphatidyl ethanolamine (DSPE). The hydrophobic chain can be either attached to one end of a polymer, or randomly distributed within the polymeric structure. According to one embodiment of the invention, the micelle is pluronic micelle. Preferably, it is pluronic micelle F127, P105, L122 or L61.

As used herein, “entrap” means that a molecule (e.g., a therapeutic molecule) is captured by the polar head or hydrophobic tail of the micelle-form nanoparticle of the invention so that the molecule exists in a monomer form.

As used herein, “water-soluble diluent” refers to an excipient dissolvable in water used as a diluent for carrying nanoparticles of the invention. When spray dried, the diluent, such as ribose, arabinose, xylose, lyxose, ribulose, xylulose, glucose, mannose, fructose, galactose, talose, allose, altrose, gulose, idose, sorbose, tagatose, maltose, sucrose, lactose, mannitol, trehalose and sorbitol (lactose and mannitol are preferred), forms respirable dry powder microparticles, each of which contains at least one nanoparticle entrapping therapeutic agent therein. The dry powder microparticles having nanoparticles with entrapped therapeutic agent can have a particle size of about 1 to about 5 microns, suitable for deep lung delivery. In addition, the size of the dry powder microparticle can be increased to target alternate delivery sites, such as the upper bronchial region or nasal mucosa by increasing the concentration of dissolved diluent in the aqueous dispersion prior to spray drying, or by increasing the droplet size generated by the spray dryer. According to one embodiment of the invention, the nanoparticle and water-soluble diluent are in a ratio ranging from 1:10 to 1:100 (w/w), preferably 1:10 to 1:60(w/w), 1:10 to 1:50(w/w), 1:10 to 1:40(w/w), 1:10 to 1:30(w/w), 1:10 to 1:20(w/w).

### B. Therapeutic Agents Entrapped in Nanoparticles

Suitable therapeutic agents include those intended for pulmonary delivery. Such pulmonary delivery is effective both for systemic delivery and for localized delivery to treat diseases of the air cavities. Preferably, the therapeutic agent is a hydrophobic drug. Preferable classes of therapeutic agents include proteins, peptides, bronchodilators, corticosteroids, elastase inhibitors, analgesics, anti-fungals, cystic-fibrosis therapeutic agents, asthma therapeutic agents, emphysema therapeutic agents, therapeutic agents of respiratory distress syndrome, therapeutic agents of chronic bronchitis, therapeutic agents of chronic obstructive pulmonary disease, therapeutics of organ-transplant rejection, therapeutic agents of tuberculosis and other infections of the lung, therapeutic agents of fungal infection, and therapeutic agents of respiratory illness associated with acquired immune deficiency syndrome, oncology therapeutic agents, therapeutic agents of systemic admiration of anti-emetics, analgesics, cardiovascular agents, photosensitizers, etc.

The therapeutic agents can be selected from a variety of known classes of drugs, including, for example, analgesics, anti-inflammatory agents, anthelmintics, anti-arrhythmic agents, antibiotics (including penicillins), anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives (hypnotics and neuroleptics), astringents, beta-adrenoceptor blocking agents, blood products and substitutes, cardiac inotropic. agents, contrast media, corticosteroids, cough suppressants (expectorants and mucolytics), diagnostic agents, diagnostic imaging agents, diuretics, dopaminergics (antiparkinsonian agents), baemostatics, immuriological agents, lipid regulating agents, muscle relaxants, parsympathomimetics, parathyroid calcitonin and biphosphonates, prostaglandins, radio-pharmaceuticals, sex hormones (including steroids), anti-allergic agents, stimulants and anoretics, sympathomimetics, thyroid agents, vasodilators and xanthines. Other therapeutic, prophylactic or diagnostic agents also can be incorporated. Examples include synthetic inorganic and organic compounds, proteins and peptides, polysaccharides and other sugars, lipids, and nucleic acid sequences having therapeutic, prophylactic or diagnostic activities. Nucleic acid sequences include genes, antisense molecules which bind to complementary DNA to inhibit transcription, and ribozymes. The agents to be incorporated can have a variety of biological activities, such as vasoactive agents, neuroactive agents, hormones, anticoaguulants, immunomodulating agents, cytotoxic agents, antibiotics, antivirals, antisense, antigens, and antibodies. In some instances, the proteins may be antibodies or antigens which otherwise would have to be administered by injection to elicit an appropriate response.

The therapeutic agents also can be photosensitizers. A photosensitizer refers to a substance which, upon irradiation with electromagnetic energy of the appropriate wavelength, usually light of the appropriate wavelength, produces a cytotoxic effect. A variety of synthetic and naturally occurring photosensitizers can be used. Many photosensitizers produce singlet oxygen. Upon electromagnetic irradiation at the proper energy level and wavelength, such a photosensitizer molecule is converted to an energized form. Singlet oxygen is highly reactive, and is toxic to a proximal target organism. Photosensitizers include, but are not limited to, hematoporphyrins, such as hematoporphyrin HCl and hematoporphyrin esters; dihematophorphyrin ester; hematoporphyrin IX and its derivatives; 3,1-meso tetrakis (o-propionamidophenyl) porphyrin; hydroporphyrins such as chlorin, herein, and bacteriochlorin of the tetra (hydroxyphenyl) porphyrin series, and synthetic diporphyrins and dichlorins; o-substituted tetraphenyl porphyrins (picket fence porphyrins); chlorin e6; monoethylendiamine monamide; mono-1-aspartyl derivative of chlorin e6, and mono- and diaspartyl derivatives of chlorin e6; the hematoporphyrin mixture Photofrin II; benzophorphyrin derivatives (BPD), including benzoporphyrin monoacid Ring A (BPD-MA), tetracyanoethylene adducts, dimethyl acetylene dicarboxylate adducts, Diels-Adler adducts, and monoacid ring “a” derivatives; a naphthalocyanine; toluidine blue O; aluminum sulfonated and disulfonated phthalocyanine ibid.; phthalocyanines without metal substituents, and with varying other substituents; a tetrasulfated derivative; sulfonated aluminum naphthalocyanines; methylene blue; nile blue; crystal violet; azure β chloride; toluidine blue; and Rose Bengal. The photosensitizer used in the invention is preferably hematoporphyrin, chlorine e6, toluidine blue, Rose Bengal, or methylene blue. Other potential photosensitizers include, but are not limited to, pheophorbides such as pyropheophorbide compounds, anthracenediones; anthrapyrazoles; aminoanthraquinone; phenoxazine dyes; phenothiazine derivatives; chalcogenapyrylium dyes including cationic selena- and tellura-pyrylium derivatives; verdins; purpurins including tin and zinc derivatives of octaethylpurpurin and etiopurpurin; benzonaphthoporphyrazines; cationic imminium salts; and tetracyclines.

An effective amount of the therapeutic amount should be included in the present dry powder microparticle. As used herein, “effective amount” refers to the amount of the therapeutic agent needed to bring about the desired result, such as achieving the intended treatment or prevention of a disorder in a patient, or regulating a physiological condition in a patient. Such an amount will therefore be understood as having a therapeutic and/or prophylactic effect on a patient. The effective amount will vary with the particular agent used, the parameters determined for the agent, the nature and severity of the disorder being treated, the patient being treated, and the route of administration. The determination of what constitutes an effective amount is well within the skill of one skilled in the art.

C. Loading of Therapeutic Agent into Micelles

Loading of one or more therapeutic agent into the micelle can be realized with techniques well known to one skilled in the art. For example, loading may be effected by dissolution of the compound in a solution containing preformed micelles, by the oil-in-water procedure or the dialysis method. Further, therapeutic agents can be incorporated into the polymeric micelle of the invention by means of chemical conjugation or by physical entrapment, emulsification techniques, simple equilibration of the agent and micelles in an aqueous medium. Hydrophilic agents such as proteins may also be incorporated into the polymeric micelles of the invention. The incorporation of such hydrophilic species may, however, require the chemical hydrophobization of the molecule or a particular affinity for the hydrophilic shell. Polyionic compounds can be incorporated through the formation of polyionic complex micelles. Physical entrapment of therapeutic agents is generally carried out by a dialysis or oil-in-water emulsion procedure. The dialysis method consists of bringing the drug and copolymer/lipid vehicle from a solvent in which they are both soluble, such as ethanol or N,N-dimethylformamide, to a solvent that is selective only for the hydrophilic part of the polymer, such as water. As the good solvent is replaced with the selective one, the hydrophobic portion of the polymer associates to form the micellar core incorporating the insoluble drug during the process. Complete removal of the organic solvent may be brought about by extending the dialysis over several days. In the oil-in-water emulsion method, a solution of the drug in a water-insoluble volatile solvent, such as chloroform, is added to an aqueous solution of the copolymer/lipid vehicle to form an oil-in-water emulsion. The micelle-therapeutic agent conjugate is formed as the solvent evaporates.

### D. Preparation of Dry Powder Microparticle

Dry powder microparticles of the invention are preferably prepared by spray drying, spray freeze drying or freeze-drying. The resulting dry powders can be further subjected to milling. Jet milling is a preferable process. In general, spray drying is a process which combines a highly dispersed liquid and a sufficient volume of a hot gas to produce evaporation and drying of the liquid droplets to produce a powder. The preparation or feedstock can be a solution, suspension, slurry, or colloidal dispersion that is atomizable. The adjustable parameters include inlet and outlet temperature, solution pump flow rate, and the aspirator partial vacuum. According to the invention, the excipient (preferably mannitol or lactose; more preferably lactose) is dissolved in aqueous solvent (such as water) and heated to increase its solubility. Then, the solution is mixed with nanoparticles as feedstock. Spray drying of a dry powder microparticle is carried out, for example, as described generally in the Spray Drying Handbook, 5.sup.th ed., (1991), j. Control. Release 70, 329-339, 2001, International Journal of pharmaceutics 269, 457-467, 2004, or Pharm. Sci. 3, 583-586, the contents of which are incorporated hereinto by reference.

Freeze-drying (also known as lyophilization or cryodesiccation) is a dehydration process typically used to preserve a material or make the material more convenient for transport. Freeze-drying works by freezing the material and then reducing the surrounding pressure and adding enough heat to allow the frozen water in the material to sublime directly from the solid phase to gas. Freeze-drying is customarily used in the preparation of nanoparticles (see Drug Development and Industrial Pharmacy (2008) iFirst, 1-6; and Journal of Pharmaceutical Sciences, Vol. 91, NO. 2, 2002, 482-491).

Spray freeze drying is a promising technique in the production of high-quality porous particles. Spray freeze dried particles have ideal aerodynamic and physical characteristics suitable for application in pulmonary drug delivery (International Journal of Pharmaceutics 319 (2006) 155-161; and International Journal of Pharmaceutics 305 (2005) 180-185.

### E. Pulmonary Delivery of Dry Powder Microparticle

Dry powder microparticles as described herein may be delivered using any suitable dry powder inhaler (DPI), i.e., an inhaler device that utilizes the patient's inhaled breath as a vehicle to transport the dry powder drug to the lungs. Preferred are Inhale Therapeutic Systems' dry powder inhalation devices as described in U.S. Pat. No. 5,458,135, U.S. Pat. No. 5,740,794, and U.S. Pat. No. 5,785,049, incorporated hereinto by reference. When administered using a device of this type, the dry powder particles containing medicaments are contained in a receptacle having a puncturable lid or other access surface, preferably a blister package or cartridge, where the receptacle may contain a single dosage unit or multiple dosage units. Convenient methods for filling large numbers of cavities (i.e., unit dose packages) with metered doses of dry powder medicament are described, e.g., in International Patent Publication WO 97/41031, incorporated hereinto by reference. Other dry powder dispersion devices for pulmonary administration of dry powders include those described, for example, in U.S. Pat. No. 3,906,950, U.S. Pat. No. 4,013,075, European Patent No. 129985, European Patent No. EP472598, European Patent No. EP 467172, and U.S. Pat. No. 5,522,385, incorporated hereinto by reference. Also suitable for delivering the antifungal dry powders of the invention are inhalation devices such as the Astra-Draco “TURBUHALER”. This type of device is described in detail in U.S. Pat. No. 4,668,218, U.S. Pat. No. 4,667,668 and U.S. Pat. No. 4,805,811, all of which are incorporated hereinto by reference. Other suitable devices include dry powder inhalers such as Rotahaler® (Glaxo), Discus® (Glaxo), Spiros® inhaler (Dura Pharmaceuticals), and the Spinhaler® (Fisons). Also suitable are devices which employ the use of a piston to provide air for either entraining powdered medicament, lifting medicament from a carrier screen by passing air through the screen, or mixing air with powder medicament in a mixing chamber with subsequent introduction of the powder to the patient through the mouthpiece of the device, such as that described in U.S. Pat. No. 5,388,572, incorporated hereinto by reference.

The dry powder microparticles of the invention can be used for lung-specific applications such as treatment for lung cancer, cystic fibrosis or asthma or system applications through the lung epithelium into the systemic circulation.

The following examples are given to illustrate the present invention. It should be understood, however, that the invention is not to be limited to the specific conditions or details described in these examples.

### EXAMPLE

### Example 1

### Preparation of Lactose Microparticle Containing Hematoporphyrin Dihydrochloride (Hp) Entrapped Micelle

### Preparation and Characterization of Hp Encapsulated in Micelle

Pluronic block copolymers, L122 (Sigma, St Louis, Mo., USA), P105 (Sigma, St Louis, Mo., USA) and F127 (Wei Ming Pharmaceutical, Taipei, Taiwan) were used in this study. Hp was entrapped into micelles with the film formation method (Photochem Photobiol. 77:299-303, 2003). Hp solution in methanol was added to the solution of L122 or F127 in chloroform, or P105 in dichloromethane to obtain 100:1, 100:2, 100:4 and 100:10 polymer/drug (wt/wt) ratios in a round bound flask so that Hp was entrapped into these copolymers. The resulting solution was heated for evaporation so that the solvent was removed and a copolymer thin film was formed after the solvent was removed. 1 ml of distilled water was added to the film at room temperature for hydration to give a final 10% w/v solution. The resulting solution was kept overnight at room temperature and then passed through a 0.2 μm PVDF filter (Millipore®, Volketswill, Switzerland) to remove the free Hp. Size distribution was measured with dynamic light scattering using a particle sizer (Coulter N4 Plus Submicron, Beckman Coulter). The solution was lyophilized to obtain freeze dried Hp entrapped micelle (micelle-Hp).

### Determination of Drug Loading and Entrapment Efficiency on Micelle

A certain amount of freeze dried Hp entrapped micelle was dissolved in absolute ethanol to extract Hp. The amount of Hp was measured with Beckman COULTER DU800 spectrophotometer with absorption at 397 nm. The drug loading and the entrapment efficiency were calculated according to the following equations (Eur J Pharm Biopharm. 55:115-124, 2003).

$$Drug loading ( \% ) = \frac{Amount of Hp in micelles}{Amount of micelles} \times 100$$

$$Entrapment efficiency ( \% ) = \frac{Drug loading}{Theoretical drug loading} \times 100 \%$$

The maximum drug loading of 7.9% was obtained in L122 micelle, 6.3% in P105 and 7.4% in F127 micelle at the ratio of polymer to Hp as 100:10. The maximum entrapment efficiency of 99.5% was obtained in L122 micelles.

### Preparation of Lactose Microparticle Containing Micelle-Hp

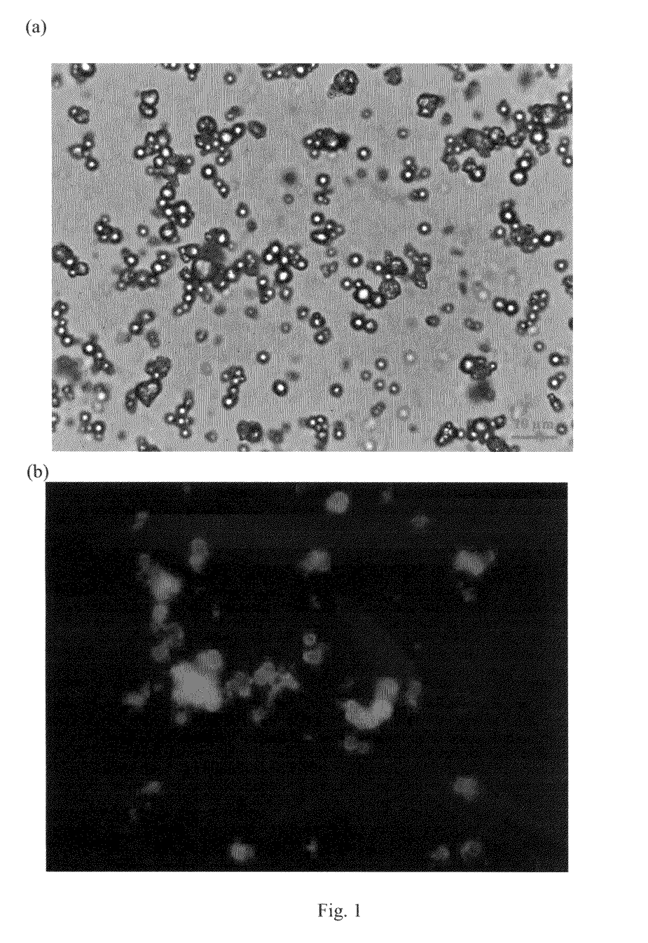

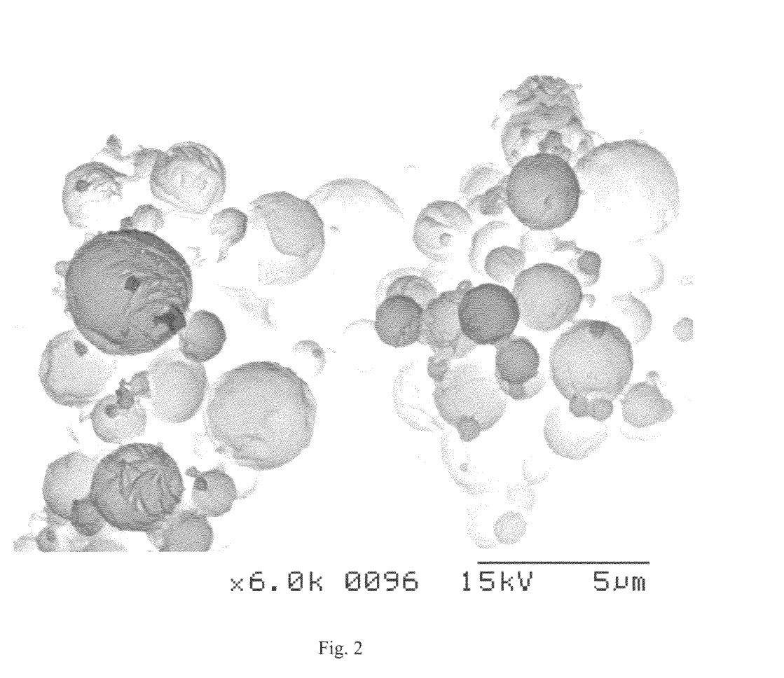

The micelle-Hp solution was pumped into the feeding system of EYELA SD-1000 spray dryer (Japan). 2 g of lactose were dissolved in 99 ml distilled water and mixed with 1 ml of 0.1 g pluronic micelle containing 2 mg Hp. The glass chambers of the spray dryer were shielded from light. The resulting powders were obtained from the collector vessel and stored at 4 under protection from light. The morphology of the spray dried powders of lactose microparticles containing L122 micelle-Hp (lactose-L122 micelle Hp) was examined using optical microscope (O-BX51), fluorescence microscope (Olympus-BX51) and scanning electron microscopy (SEM; Hitachi, S-2700, Japan). The microparticles were shown to be spherical (FIG. 1a). FIG. 1b shows fluorescence microscope plot of the lactose-L122 micelle Hp. It can be seen that Hp was successfully entrapped into micelle L122 and the resulting L122 micelle-Hp was evenly distributed in the lactose carrier (red fluorescence). Moreover, FIG. 2 shows the SEM plot for the surface of morphology of lactose-L122 micelle Hp. The mean geometric particle size of lactose-L122 micelle Hp is 2.3±0.6 μm, so it is appropriate for maximizing pulmonary deposition of dry powders (representing the deep lungs).

### Example 2

### Size and Solubility of Micelle after Spray-Drying

The mean particle size of L122 micelle Hp was measured before and after spray-drying (L122: lactose=1:0, without spray-drying) and it showed that the particle size of L122 micelle Hp was not significantly changed after spray-drying and re-dissolving in the water.

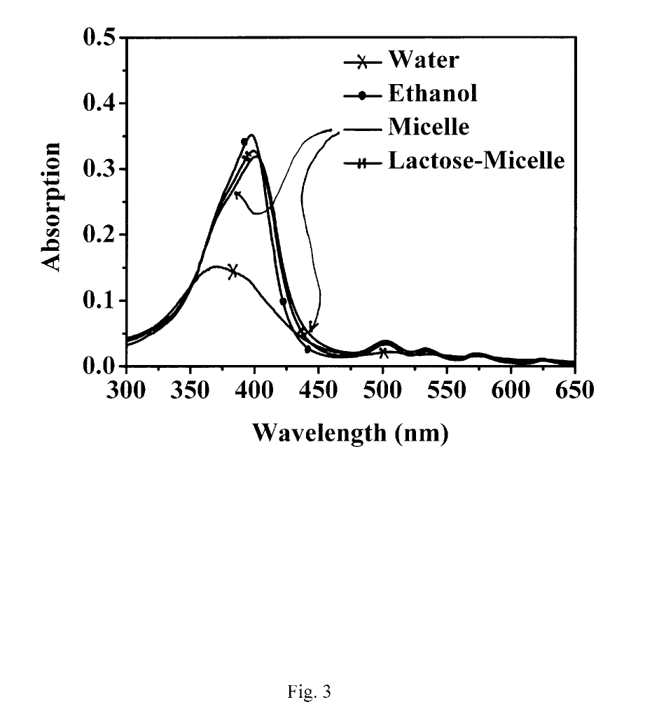

The maximum wavelength of the absorption band (λₘₐₓ) of Hp was measured for Hp, L122 micelle Hp and lactose-L122 micelle Hp after they were re-dissolved in the water (Beckman COULTER DU800 spectrophotometer). The maximum absorption peak of L122 micelle Hp was shown at 398 nm and a similar pattern could be observed when Hp dissolved in ethanol. After spray-drying, the maximum absorption peak of lactose-L122 micelle Hp did not change. The results shown in FIG. 3 indicated that the Hp remained in a monomer form after spray-drying. The table below shows particle size of micelle-Hp and lactose-micelle Hp and the values of λₘₐₓ, λ₃₉₇/λ₃₇₂.

| | Particle size^{e} | | |
|---|---|---|---|
| | (nm) | λₘₐₓ | λ₃₉₇/λ₃₉₂^{f} |
| L122 micelle Hp^{a} | 105 ± 30 | 398 | 1.351 ± 0.001 |
| Lactose-L122 micelle Hp^{b} | 112 ± 46 | 398 | 1.359 ± 0.007 |
| Mannitol-L122 micelle Hp^{b} | 279 ± 117 | | |
| L61 micelle^{a} | 231 ± 37 | | |
| Lactose-L61 micelle^{b} | 343 ± 199 | | |
| Hp in PBS^{c} | | 372 | 0.831 ± 0.037 |
| Hp in ethanol^{d} | | 397 | 1.428 ± 0.014 |

^{a}Before spray drying
^{b}After spray-drying and re-dissolving in water
^{c}Free Hp dissolved in PBS
^{d}Free Hp dissolved in ethanol
^{e}Micelle size
^{f}Ratio of monomer to dimer (absorbance at 397 nm/372 nm).

The relative absorption intensity of the Hp in monomer form (at 397 nm) and aggregated form (at 372 nm) can be used as a measure of the aggregation of Hp in solution (N. Hioka et al., 80:1321-1326, 2002). The λ₃₉₇/λ₃₇₂ ratio is similar to when Hp in ethanol (1.41) is higher than Hp in PBS (0.78), indicating that a higher level of monomerization of Hp occurred after spray-drying and re-dissolving in the water. This result indicates that the photochemical properties of Hp entrapped in micelle after spray-drying with lactose and re-dissolving carrier particles in the water are not changed.

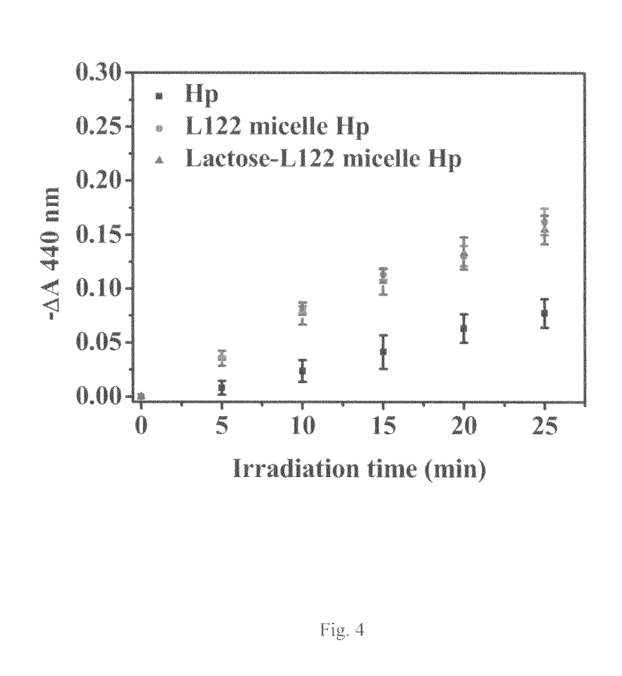

The generation of singlet oxygen in the presence of histidine for Hp in PBS, L122 micelle Hp in PBS and the spray-dried Lactose-L122 micelle Hp that was re-dissolved in PBS was detected by spectrophotometric measurement of p-nitroso-dimethylaniline (RNO) bleaching, induced by imidazole as a singlet oxygen specific substrate. The singlet oxygen was generated by illuminating HP, L122 micelle Hp and Lactose-L122 micelle Hp, and it reacted with histidine to form a transannular peroxide product. This product rendered RNO bleaching and an absorbance can be observed at 440 nm. As shown in FIG. 4, there are no significant differences between the rates of RNO photobleaching in L122 micelle Hp and Lactose-L122 micelle Hp, which demonstrates that the micelle-Hp maintains the original activity after it is entrapped with lactose, spray-dried and re-dissolved in PBS. The differences between Hp in PBS and in micelle or in lactose-micelle are significant.

If micelle is disintegrated after it is entrapped with lactose, spray-dried and then re-dissolved in PBS, the Hp will aggregate and reach an excited state in an aqueous medium through a self-quenching effect (S. A. Gerhardt et al., Journal of Physical Chemistry A. 107:2763-2767, 2003). As can be seen from prior art references, aggregated photosensitizers generally produce very little ¹O₂ and have much lower photodynamic activity. In this study, after re-dissolving the spray-dried Lactose-L122 micelle Hp, the λmax and λ397/λ372 ratio of Hp was similar to that in ethanol. Furthermore, oxygen consumption experiments indicate that after the spray-dried lactose microparticles are re-dissolved, the micelle is not broken and the high levels of monomer Hp remain in micelle.

### Example 3

### Cellular Uptake of Free Hp, L122 Micelle Hp and Lactose-L122 Micelle Hp

Human lung adenocarcinoma A549 cells were kept in a humidified incubator containing 5% CO₂ at 37° C. A549 cells were cultured in DMEM supplemented with 10% fetal bovine serum (FBS) and 1% penicillin-streptomycin (GIBCO BRL, USA). The cells were routinely grown in tissue culture flask and harvested with a solution of 1% trypsin while in the logarithmic phase of growth. The cells were kept in the above culture conditions for experiments.

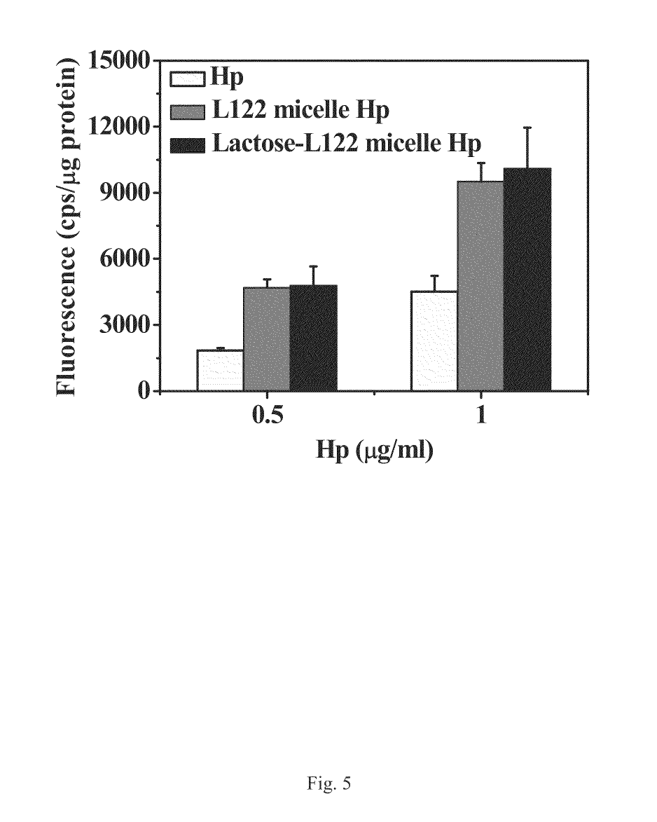

A549 cells were seeded in a 6 well plate at 2×10⁵ cell per well (2 ml cell suspension) and incubated at 37° C. under a 5% CO₂ atmosphere for 24 hours. The medium was removed and 2 ml DMEM media containing free Hp, L122 micelle Hp or lactose-L122 micelle Hp were added to different wells for incubating cells at 37° C. under a 5% CO₂ atmosphere for 3 hours. Subsequently, the medium was removed and the cells were washed twice with 2 ml PBS. 1 ml lysis buffer (0.1 N NaOH) was added followed by incubation on ice for 10 min to lyse the cells. The resulting solution was homogenized and centrifuged at 14000 rpm for 20 min. The fluorescence of the supernatant was measured using a spectrophotometer (Ex: 397 nm, Em: 633 nm). 25 μl of the cell lysates were used in the MicroBCA™ protein assay. The uptake of Hp was calculated as fluorescence per μg of cellular protein. FIG. 5 shows the uptakes of L122 micelle Hp and lactose-L122 micelle Hp by A549 tumor cells in comparison with free Hp. The results show that the uptake of each Hp formulations by the cells is in a concentration dependent manner. The fluorescence intensities measured on A549 cells treated with L122 micelle Hp and Lactose-L122 micelle Hp were at least two-fold higher than on those treated with free Hp.

### Example 4

### Photocytotoxicity of Free Hp, L122 Micelle Hp and Lactose-L122 Micelle Hp

A549 cells were grown in 96-well plates at a density of 8×10³ cells/well for 24 hours. The culture medium was removed and DMEM medium containing free Hp, L122 micelle Hp or lactose-L122 micelle Hp (100 μl/well) was added to different wells. The cells were incubated for 3 hours (protection from light) and washed once with 100 μl PBS/well. The no phenol red medium (100 μl/well) was added to the cells and then irradiated with various doses of light using LED (635±5 nm, 60 mW/cm²) light source. After light irradiation, the original medium was removed and DMEM containing 10% FBS was added to each well. Twenty-four hours later, cell survival was measured using an MTT [3(4,5-dimethyl-thiazoyl-2-yl) 2,5 diphenyl-tetrazolium bromide] assay. The MTT assay was based on the activity of mitochondria dehydrogenases wherein a water-soluble tetrazolium salt was reduced to a purple insoluble formazan product. The amount of MTT formazan product was analyzed with spectrophotometer at the absorbance of 570 nm.

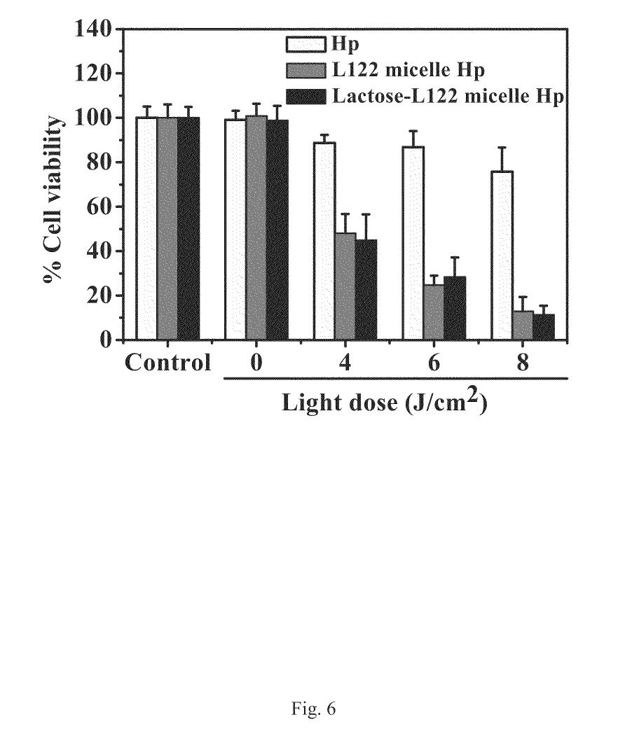

FIG. 6 shows photocytotoxicity of free Hp, L122 micelle Hp and Lactose-L122 micelle Hp on A549 cells. Kept in the dark, none of the above Hp formulations had a cytotoxic effect. After A549 cells were incubated with the above formulations with 0.5 μg/ml Hp for 3 hours and then irradiation at 4 J/cm2, 89% cells were alive in free Hp, 47% in L122 micelle Hp, and 44% in Lactose-L122 micelle Hp. After irradiation at 12 J/cm2, 75% cells were alive in free Hp, 12% in L122 micelle Hp, 11% in Lactose-L122 micelle Hp.

### Example 5

### Preparation of Lactose Microparticle Containing Rifampicin Entrapped L122 Micelle

In this study, L122 and rifampicin (RP) in a ratio of 100:1 (w/w) and 2% lactose were used in the preparation of microparticles (L122 micelle RP). The preparation process is the same as that stated in Example 1. The mean particle size of L122 micelle RP was measured before and after spray-drying and the results are listed in the table below:

| | Particle size (nm) |
|---|---|
| L122 micelle RP | 126 ± 44 |
| Lactose-L122 micelle RP | 126 ± 55 |

What is claimed is:

1. A dry powder microparticle for pulmonary delivery, which comprises at least one nanoparticle in the form of micelle, wherein the nanopaparticle entraps one or more therapeutic agents therein, and a water-soluble diluent layer surrounds the nanoparticle.

2. The dry powder microparticle of claim 1, which is of a size ranging from 1 to 10 μm.

3. The dry powder microparticle of claim 2, which is of a size ranging from 1 to 5 μm, 5 to 8 μm or 8 to 10 μm.

4. The dry powder microparticle of claim 1, wherein the nanoparticle is of a size ranging from 3 to 1000 nm.

5. The dry powder microparticle of claim 1, wherein the nanoparticle is of a size ranging from 3 to 700 nm, 3 to 500 nm, 3-300 nm, 3-150 nm, 3-110 nm, 3-100 nm, 3-50 nm, 3-30 nm, 50-100 nm, 50-200 nm, 50-300 nm, 50-500 nm or 50-700 nm.

6. The dry powder microparticle of claim 1, wherein the nanoparticle is of a size ranging from 3 to 150 nm.

7. The dry powder microparticle of claim 1, wherein the nanoparticle is of a size ranging from 3 to 110 nm.

8. The dry powder microparticle of claim 1, wherein the nanoparticle is of a size ranging from 3 to 50 nm.

9. The dry powder microparticle of claim 1, wherein the nanoparticle is of a size ranging from 3 to 30 nm.

10. The dry powder microparticle of claim 1, wherein the nanoparticle and water-soluble diluent are in a ratio ranging from 1:10 to 1:60 (w/w).

11. The dry powder microparticle of claim 1, wherein the nanoparticle and water-soluble diluent are in a ratio ranging from 1:10 to 1:50(w/w), 1:10 to 1:40(w/w), 1:10 to 1:30(w/w) or 1:10 to 1:20(w/w).

12. The dry powder microparticle of claim 1, wherein the micelle is normal micelle, reverse micelle, polymeric micelle or pluronic micelle.

13. The dry powder microparticle of claim 12, wherein the pluronic micelle is F127, P105, L122 or L61.

14. The dry powder microparticle of claim 12, wherein the therapeutic agent is a hydrophobic drug.

15. The dry powder microparticle of claim 1, wherein the therapeutic agent is selected from the group consisting of: proteins, peptides, bronchodilators, corticosteroids, elastase inhibitors, analgesics, anti-fungals, cystic-fibrosis therapeutic agents, asthma therapeutic agents, emphysema therapeutic agents, therapeutic agents of respiratory distress syndrome, therapeutic agents of chronic bronchitis, therapeutic agents of chronic obstructive pulmonary disease, therapeutics of organ-transplant rejection, therapeutic agents of tuberculosis and other infections of the lung, therapeutic agents of fungal infection, and therapeutic agents of respiratory illness associated with acquired immune deficiency syndrome, oncology therapeutic agents, therapeutic agents of systemic admiration of anti-emetics, analgesics, cardiovascular agents and photosensitizers.

16. The dry powder microparticle of claim 12, wherein the photosensitizer is selected from the group consisting of: hematoporphyrins, 3,1-meso tetrakis (o-propionamidophenyl) porphyrin, hydroporphyrins, chlorin e6 monoethylendiamine monamide, the hematoporphyrin mixture Photofrin II, benzophorphyrin derivatives, tetracyanoethylene adducts, dimethyl acetylene dicarboxylate adducts, Diels-Adler adducts, a naphthalocyanine, toluidine blue O, aluminum sulfonated and disulfonated phthalocyanine ibid, a tetrasulfated derivative, sulfonated aluminum naphthalocyanines, methylene blue, nile blue; crystal violet; azure β chloride, toluidine blue, chlorine e6, and Rose Bengal.

17. The dry powder microparticle of claim 16, wherein the photosensitizer is selected from the group consisting of hematoporphyrin, methylene blue, toluidine blue, chlorine e6 and Rose Bengal.

18. The dry powder microparticle of claim 1, wherein the diluent is ribose, arabinose, xylose, lyxose, ribulose, xylulose, glucose, mannose, fructose, galactose, talose, allose, altrose, gulose, idose, sorbose, tagatose, maltose, sucrose, lactose, mannitol, trehalose or sorbitol.

19. The dry powder microparticle of claim 1, wherein the diluent is lactose or mannitol.
